(19) United States
(12) Patent Application Publication
(10) Pub. No.: US 2010/0167403 A1
(43) Pub. Date: Jul. 1, 2010

(54) IDENTIFICATION AND SELECTION OF COMMERCIAL-QUALITY TRANSGENIC PLANT EVENTS

(75) Inventor: JAMES C. REGISTER, III, Ames, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 12/346,558

(22) Filed: Dec. 30, 2008

(51) Int. Cl. C12N 5/10; G01N 33/48; G06Q 99/00

(52) U.S. Cl. 435/419; 702/19; 705/500

(57) ABSTRACT

A method for identifying and selecting commercial-quality transgenic plant event from a set of transgenic plant events includes determining a desired set of characteristics for the product, identifying data for assessing the desired set of characteristics, forming a staged experimental plan for generating the data, and executing the staged experimental plan to select at least one event for commercialization.

### FIELD OF THE INVENTION

The present invention relates to, but is not limited to, identification and selection of commercial-quality transgenic plant events.

### BACKGROUND

Transgenic plant events have been commercialized in various ways. Typically, significant resources are expended in identifying and selecting events which are believed to be of interest and can be commercialized. Conventionally, such resources are allocated on a largely ad hoc basis. Such an approach is understandable given the nature of biological material.

The biotechnological problem is that multiple transgenic plant events created using the same genetic construct will not be identical. Plant genomes are very large, and in different events the transgene will have inserted at different locations within the genome. Location will, in ways subtle or less so, influence how and when the transgene is expressed. Furthermore, because the methods for introducing transgenes into a plant cell are not always precise, each event may have slight differences in the composition of the insertion, ranging from gross differences such as copy number or significant rearrangements of the transgene to more subtle imperfections. These differences may also impact how or when a gene is expressed. It is also possible that the location or nature of the insertion will affect endogenous genes in the plants, making changes in the expression of those genes. Some of those changes may be observable only under particular growth conditions or under the influence of various types of stress. Thus, events will differ both in the efficacy of the transgene and the overall agronomic performance of the line derived from the event. Therefore, the skilled artisan may need to create tens to thousands of events with a given construct to identify a single event that possesses all desired qualities for commercialization. The greater the event number, the greater the potential cost and complexity of determining which if any of the events possess all the desired qualities.

From a business perspective, what is needed is a way to increase process efficiency and cost-effectiveness.

### SUMMARY OF THE INVENTION

A method for identifying and selecting commercial-quality transgenic plant event from a set of transgenic plant events includes determining a desired set of characteristics for the product, identifying data for assessing the desired set of characteristics, forming a staged experimental plan for generating the data, and executing the staged experimental plan to select at least one event for commercialization.

### BRIEF DESCRIPTION OF THE DRAWINGS

FIG. 1 is a flow chart illustrating a methodology.

FIG. 2 is a diagram illustrating a determination of a desired set of characteristics.

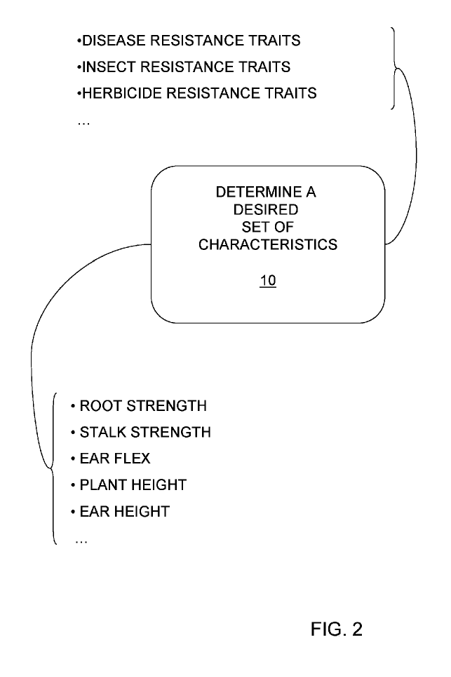

FIG. 3 is a diagram illustrating a determination of data to be used in assessing a desired set of characteristic.

FIG. 4 illustrates a formation of a staged experimental plan.

FIG. 5 illustrates formation of a staged experimental plan in accordance with example 1.

FIG. 6 illustrates formation of a staged experimental plan in accordance with example 2.

### DETAILED DESCRIPTION

A methodology is provided which provides a systematic approach to the characterization and selection of commercial-quality transgenic plant events. The methodology allows for steps associated with the commercialization of transgenic plants to be organized or arranged in a manner which allows for efficient collection of data and use of resources.

### 1. Overview

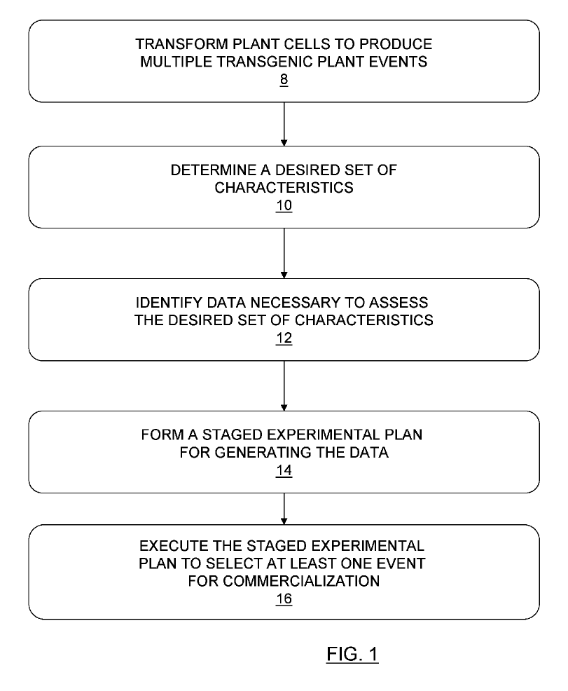

FIG. 1 is a flow chart illustrating one methodology. In FIG. 1, in step 8 plant cells are transformed to produce multiple transgenic plant events. In step 10, a desired set of characteristics is selected. The desired set of characteristics may include characteristics associated with or believed to be necessary for successful commercialization. It is to be understood that the steps need not necessarily occur in the order shown in FIG. 1. For example the desired set of characteristics may be determined prior to the production of multiple transgenic plant events, and one or more of these characteristics may be used to drive the transformation process in whole or in part.

In step 12, a determination is made with respect to the data necessary to assess the desired set of characteristics. Typically such data may include an assessment of the phenotype resulting directly from expression of one or more transgenes as well as characteristics of the transgenic events and should include evaluations across multiple plant genotypes and environments.

In step 14, a staged experimental plan is formed. The staged experimental plan will, when executed, generate the data. A staged experimental plan may include various stages for measurements or evaluations of the whole plant, physiologic, molecular or biochemical evaluations. The staged experimental plan may also include steps for obtaining assessments needed for or otherwise associated with regulatory approval.

The staged experimental plan is preferably staged in a manner which provides for efficient allocation of resources. It is to be understood that selection of transgenic events is an exclusionary process where a set of candidate transgenic events is reduced by subjecting the candidates to various experiments and by excluding those candidates which fail or perform undesirably. There are costs which may be associated with each experiment. In addition, each experiment may have a varying degree of effectiveness in eliminating candidates. As will be later explained in greater detail, there may be other constraints associated with various tests such as, but not limited to, the amount or type of biological material needed or the amount of time required to perform the test. As will also later be discussed, it is to be understood that although a staged experimental plan is formed prior to its execution, the results from prior stages in an experimental plan may be used to affect the organization of future stages. Thus, a staged experimental may allow for branching based on results obtained in previous stages.

In step 16, the staged experimental plan is executed to select at least one event for commercialization.

### 1.1. Transform Plant Cells to Produce Multiple Transgenic Plant Events

Any method may be used to introduce a polynucleotide into a plant cell. Such protocols may vary depending on the type of organism, cell, plant, or plant cell, i.e. monocot or dicot, used for transformation. Suitable methods of transforming plant cells that can used alone or in combination include microinjection, electroporation, viral-mediated transformation, chemical-mediated delivery (e.g., PEG methods), bacterial-mediated methods (e.g., Agrobacterium-mediated, Rhizogenes-mediated), sonication, and particle-mediated delivery (e.g., ballistic methods, and silicon carbide whisker methods) (see, e.g., Rakoczy-Trojanowska (2002) Cell Mol Biol Lett 7:849-858; and Birch (1997) Annu Rev Plant Physiol Plant Mol Biol 48:297-326). Typically, a screenable marker system is used to identify transformed plant cells. If the polynucleotide does not include a screenable marker, another polynucleotide containing the screenable marker can be delivered to produce transient or stable expression of the marker.

The cells having the introduced sequence may be grown or regenerated into plants using conventional conditions, see, for example, McCormick, et al., (1986) Plant Cell Rep 5:81-4. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide is stably maintained and inherited, and seeds harvested.

### 1.2. Desired Characteristics

The desired characteristics may include particular traits of interest associated with one or more transgene(s), such as, but not limited to agronomic traits, disease resistant traits, insect resistant traits, herbicide tolerance traits, tolerance to herbicide-resistant weeds, efficient nitrogen use, nutritional enhancements, firmness, acidity content, sugar content, texture, oil, starch, carbohydrate, or nutrient metabolism, increased oil production, increased protein production, unique oil and protein production, increased fermentable starch production, increased content of essential amino acids, increased content of fatty acids, modified flower senescence, improved traits related to commercial processing, enhanced digestibility, sterility, grain characteristics, or improved rate of growth. It is to be understood that a transgenic plant may have two or more stacked traits and that the traits may be endogenous or transgenic or a combination thereof to the plant.

Examples of agronomic traits include yield, yield stability, abiotic stress tolerance, drought tolerance, cold stress tolerance, heat stress tolerance, stalk qualities, root mass, canopy characteristics, lodging, nitrogen utilization, seed set, seed size, plant height, ear height, and the like. Herbicide resistance traits include resistance to herbicides that act to inhibit the action of acetolactate synthase (ALS), such as sulfonylurea-type herbicides, resistance to herbicides that act to inhibit action of glutamine synthase, such as phosphinothricin or bialaphos, resistance to herbicides in the hydroxyphenyl pyruvate dioxygenase (HPPD)-inhibiting family, and resistance to glyphosate. Sterility, such as male sterility is another trait of interest.

Insect resistance genes may encode resistance to pests such as rootworm, cutworm, armyworm, earworm, European Corn Borer, and the like. Such genes include Bacillus thuringiensis (Bt) toxic protein genes. Disease resistance traits include resistance or tolerance against to Fusarium spp., Phytopthera spp., Pseudomonas spp., Erwinia spp., Gibberella spp., Sclerotinia spp., anthracnose, white mold, stalk rots, root rots, ear molds, leaf spots, rust, blights, mildews, nematodes, and/or viral diseases.

Male sterility can be conferred by genes including male tissue-preferred genes and genes with male sterility phenotypes such as QM. Other genes include kinases and those encoding compounds toxic to either male or female gametophytes. Examples of seed or grain quality traits include traits such as levels and types of oils, saturated and unsaturated, amino acid composition and/or levels, starch composition and/or levels, wet or dry milling properties, and/or end products.

The transgenic plant may include, but is not limited to, corn, soybean, sorghum, cotton, rice, wheat, canola, vegetable crops, forest tree crops, forage crops, ornamental crops, turf grasses and cellulosic ethanol feedstocks.

Where the desired characteristics, are achieved by transgenic plant events, the expression of these genes in transgenic crop plants should not reduce the vigor, viability or fertility of the plants, nor should it affect the normal morphology of the plants in ways unrelated to the transgene(s). Expression of such genes in transgenic crops should not interfere with the recovery and propagation of transgenic plants, impede the development of mature plants, or confer unacceptable agronomic characteristics. Thus, the desired characteristics may include other traits as well. Thus, it is to be understood that the desired characteristics are not limited to particular traits achieved by transgenes, but may include any number of other characteristics desired for a product. Examples of characteristics associated with corn include relative maturity, yield, drought tolerance, root strength, stalk strength, stress emergence, ear flex, plant height, ear height, staygreen, grain drydown, and susceptibility to a variety of pests, including gray leaf spot, northern leaf blight, southern leaf blight, anthracnose stalk rot, head smut, European corn borer and corn rootworm.

Examples of characteristics associated with soybeans include relative maturity, yield, standability, field emergence, canopy width, shattering, percent protein at 12 percent moisture, percent oil at 13 percent moisture, iron deficiency chlorosis, and susceptibility to a variety of pests including phytophthora, brown stem rot, white mold, sudden death syndrome, cyst or root-knot nematodes, and aphid antibiosis.

Examples of characteristics associated with wheat may include drought tolerance, head type, height, leaf blight, leaf rust, lodging resistance, powdery mildew, scab, soil-borne mosaic virus, test weight, winter hardiness, and yield.

Examples of product characteristics for tomatoes may include relative maturity, fruit size, fruit shape, exterior color, as well as disease resistant characteristics such as Fusarium wilt resistance, root knot resistance, gray leaf spot resistances, tomato spotted wilt resistances, and verticillium wilt resistances.

Examples of product characteristics for pumpkins may include relative maturity, fruit height, fruit diameter, fruit weight, fruit shape, and exterior color. Product characteristics may include disease resistant traits such as powdery mildew resistance.

Examples of product characteristics for cauliflower may include relative maturity, plant habit, plant vigor, plant wrapping, head size, head shape, and color.

### 1.3. Data Necessary to Assess the Desired Characteristics

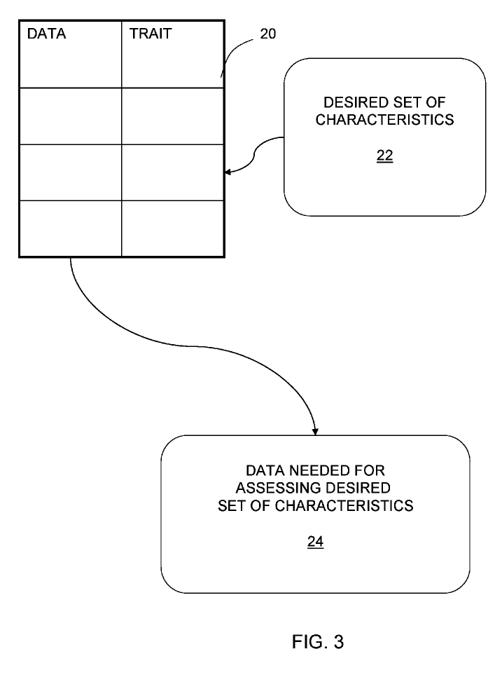

As previously explained, in step 12, a determination is made with respect to what data are necessary or otherwise desirable to assess the desired characteristics. Various methods of making such a determination are contemplated. For example, in FIG. 3, a lookup table 20 is shown. The lookup table 20 is populated with traits or characteristics and corresponding types of data which may be used to assess the presence, absence, or degree or level of the desired characteristics. The types of data may be associated with one or more tests, assays, or experiments. It is to be understood that there may be any number of alternative tests, assays, or experiments which may be used to generate data sufficient to assess any particular characteristic.

As shown in FIG. 3, a desired set of characteristics 22 is used, in conjunction with a lookup table 20, or otherwise, to determine the data which is needed, or otherwise useful, for assessing the desired set of characteristics 24. A computer may be used to assist in the process, where an input to the computer includes specification of the characteristics 22 of interest and an output indicates what data is needed for assessing the desired set of characteristics 24. Any number of applications may be used including, but not limited to, database applications and spreadsheet applications.

Instead of or in addition to using a lookup table, a user may specify the tests to be used to assess desired characteristics, or the alternative tests that may be considered.

Table I below sets forth examples of desired characteristics as well as the data necessary or otherwise desirable in assessing desired characteristics. The examples set forth below are merely illustrative, and not limiting. One skilled in the art will appreciate that there are numerous desired characteristics which will vary by species of plant, and commercialization considerations. Moreover, one skilled in the art will appreciate that there are numerous tests, assays, or experiments which may be used to generate data sufficient to assess for various characteristics.

Note: for all of the tests below, of course one excepts from “normal behavior” those phenotypes intended to be altered by the transgene.

TABLE I

| Characteristics or Trait | Type of assay and data |
|---|---|
| Copy number of insert | Quantitative PCR and/or Southern analysis. |
| Intactness of insert | Southern analysis and/or DNA sequence. |
| Lack of undesired open reading frames | Sequence information on junction regions between |
| at edges of insert | transgene insertion and plant genome. |
| Lack of undesired | PCR analysis, Southern analysis and/or DNA |
| rearrangements within insert | sequence. |
| Expression of inserted transgene | RT-PCR, Northern blot, Western immunoassay, |
| at mRNA and protein levels | ELISA and/or mass spectrometry. |
| Protein that accumulates is as | Protein sequence, enzyme or other activity |
| predicted. | assay(s), and/or a variety of methods to |
| | characterize possible modifications such as |
| | glycosylation. |
| Transgene function | Enzyme assays, activity assays, herbicide |
| | tolerance, insect feeding trials, disease tolerance |
| | scoring, drought tolerance, cold tolerance, cold |
| | germination, RT-PCR, Northern blot, oil content, |
| | oil composition, starch assay, phytate assay, mass |
| | spectroscopy, wet milling test, dry milling test, |
| | animal feeding trials, in vitro digestibility assay, |
| | amino acid composition, total protein analysis, |
| | nitrogen utilization test, metabolic profiling, |
| | expression profiling, photosynthetic assays, root |
| | mass, stalk strength, ear height, pollen shed, pollen |
| | formation, allergenicity modeling, allergenicity test |
| | panels. |
| Reduced expression of targeted | RT-PCR and/or Northern analysis. |
| endogenous gene (down- | |
| regulation) | |
| General plant health | Observations under various growth conditions, |
| | metabolic profiling. |
| Agronomic performance | Multisite and location field trials of the event(s) in |
| | different genetic backgrounds as appropriate, |
| | exposing the lines to different conditions and |
| | measuring, among other things, germination rate |
| | and vigor, growth rate, flowering time, time to |
| | maturity and the ability to withstand abiotic (heat, |
| | drought, cold, excess water, other) and biotic |
| | (insect, fungal, nematode and other pest attack) |
| | stresses. |
| Consistency of yield | Multi-location multi-season yield trials |
| Lack of weediness | Field trials as per government regulations to ensure |
| | that the transgenic line behaves normally and will |
| | not spread beyond fields or otherwise behave as a |
| | weed. |
| Normal quality of plant product | Depending on the plant species and product |
| | derived from it, test to ensure the normal taste, |
| | processing quality, viscosity, nutritional |
| | composition and quality, fiber characteristics and |
| | any other quality normally associated with the |
| | grain, fruit, vegetable, fiber or other product |
| | produced from the crop. |

### 1.4. Staged Experimental Plan

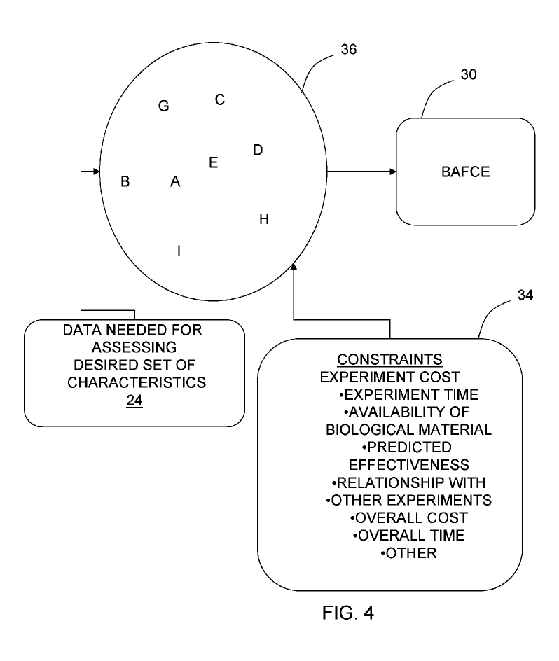

As shown in FIG. 4, a staged experimental plan 30 is formed. The staged experimental plan provides an ordering or arrangement of experiments in a manner that assists in optimizing efficiency of the overall staged experimental plan 30. A set of possible experiments 36 are shown which may be used to provide the data needed to evaluate the desired set of characteristics. The construction of a staged experimental plan 30 involves selecting appropriate experiments from the set of possible experiments, and ordering or arranging the set of experiments in a manner to assist in meeting or optimizing one or more desired objectives. As shown in FIG. 4, not all possible experiments 36 become a part of the staged experimental plan 30.

The staged experimental plan 30 is formed using the identified data necessary or useful for assessing the desired set of characteristics 24. In addition, the staged experimental plan 30 may take into consideration any number of constraints 34. The constraints 34 may include constraints associated with an experiment, test, analysis, or assay. Alternatively constraints 34 may be associated with an overall process which may include multiple experiments. Each constraint typically relates to the amount of a particular resource that may be used.

Examples of constraints 34 may include, without limitation, overall cost, overall time, or other constraints, such as experiment cost, experiment time, biological material necessary to perform an experiment, predicted effectiveness of an experiment, constraints imposed by a relationship between an experiment and other experiments, or other constraints. Thus, constraints may include pre-conditions for performing an experiment (such as availability of a particular amount of biological material) as well as the resources associated with performing a particular experiment (such as, without limitation time, cost, etc.). Each experiment which is to be performed may have a cost associated with it. The cost may be specified in any number of ways. For example, the cost may be specified as a per experiment cost, a per construct cost, a per trial cost, a per assay cost, a per plant cost, a per event cost, a batch cost, product cost, unit cost, space costs (e.g., field or greenhouse), or a per sample cost associated with performing an experiment. Or else the cost may be otherwise specified. For example, an experiment may have a fixed cost in addition to a per plant cost, per event cost, or per sample cost. It is to be further understood that in some cases it is preferable to perform multiple trials of a particular test in order to provide more accurate results. In such instances, the cost of the multiple trials may be considered. The cost associated with experiments may also include costs associated with allocation of personnel, particular laboratories, laboratory equipment, greenhouse space, field space, or other costs.

In addition, the experiments may have time related constraints associated with them. These include the time it takes to conduct to the experiment. In some cases experiments may be performed in-house. In other cases, the experiments may require outside testing which may increase the amount of time for conducting the experiment.

Predicted effectiveness is a measure of the predicted ability of an experiment to reduce the number of candidate transgenic events remaining in the set being evaluated. The predicted effectiveness may be represented with a numeric value or score. It is contemplated that the predicted effectiveness may be specified as a range. It is to be further understood that the predicted effectiveness of an experiment may be affected by when the experiment is performed relative to other experiments.

The biological material necessary to perform an experiment may also be an important consideration. Certain experiments may require more biological material than other experiments; this may impose limitations on when a particular experiment can be performed. For example, an experiment which requires a substantial amount of biological material would likely not be performed early on as sufficient biological material may simply not be available. For example, yield measurements would require large amounts of uniform seed for replicated trials. In some experiments such as molecular characterizations only small amounts of biological materials need be used from one or a few plants. It is further to be understood that lesser amounts of biological materials can be used in some instances, but using lesser amounts of biological material or performing fewer trials may compromise the accuracy of any resulting test results.

It is also to be understood that there may be constraints imposed by relationships between an experiment and other experiments. For example yield is a characteristic which has a relationship with other characteristics such as insect pressure, herbicide treatment, defined stress conditions, tolerance to defined stress condition. By way of further example, tolerance to a defined stress condition may be related to herbicide treatment. It is to be further understood that other constraints related to other resources may also be imposed. One way to categorize constraints is to distinguish between local constraints which apply, for example, to the constraints associated with a particular experiment, and global constraints which are associated with the overall use of resources for the staged experimental plan.

1.4.1

The staged experimental plan can be formed in various ways. For example, an optimization algorithm may be used to formulate an optimized arrangement of the stages of the experimental plan based on desired objectives such as lowest cost, or least amount of time. Examples of optimization algorithms which may be used include mixed integer linear programming algorithms, evolutionary algorithms such as genetic algorithms, and other types of algorithms. Alternatively, a cost/impact estimation model may be used, or stochastic models, Monte Carlo simulations, and other types of models may be used. Of course, simpler algorithms may be used.

1.4.2

It is to be further understood that the staged experimental plan 30 may include branching based on decision points in the process. Thus, the arrangement of experimental stages within the experimental plan may be based on results obtained from previous stages. Thus, the formation of an optimized experimental plan may be a dynamic process.

The staged experimental plan may be formed in a manner which contemplates different results in different stages so that what-if scenarios may be contemplated prior to the execution of the staged experimental plan. Statistical data associated with the tests or with the relevant genetics or estimations made by a user may be used to make predictions regarding the experimental process, prior to the experimental process being performed. Such predictions may be assigned probabilities to thereby assist in management of resources.

It is to be understood, however, that the staged experimental plan may include an arrangement of stages and decision points (sometimes known as gates) arranged in a manner that will allow for various different routes to be traversed before arriving at transgenic plant events to commercialize. The decision made at any particular gate may be based on results obtained in previous steps. Thus, the process of the staged experimental plan may be dynamic in nature. However, it is to be further understood that in some cases the application of constraints may limit the ability of the process to be dynamic.

### 1.5 Execution of the Staged Experimental Plan

The staged experiment plan is executed to identify and select commercial-quality transgenic plant event from a set of transgenic plant events. The staged experimental plan is dynamic in nature in that the outcome of one stage may be based on results obtained in previous steps.

### EXAMPLE 1

In this example, a simple cost/impact estimation model is provided. The model can be stated as an Assessment Valuation Index (AVI) being the product of the cost to conduct an assessment (or stage) and the expected impact on event selection of the results of the assessment. The expected impact may be stated as a percent reduction in the number of events under consideration. Thus:

$$AVI = \frac{( Impact )}{( Cost ) ( \# of Events in Pool )}$$

Impact can be quantified as the percentage of the total pool of events that can be expected to be removed based on the results from any particular assessment (or stage in the experimental plan). Assuming necessary material is available for the desired assessment(s), the approach/assay with the higher AVI should be either be applied sooner during event selection or preferentially applied versus one or more alternative approaches/assays. There are many ways such a model may be used.

For example, one may wish to eliminate from a pool of 500 events at the T0 generation any events not expressing a transgene of interest. Three methods are commonly available for this assessment—ELISA, Western immunoassay and RT-PCR. If we assume that appropriate material is available for any of the 3 alternatives, it is expected that 30% of the events do not accumulate the protein of interest (as detected by either ELISA or Western immunoassay), that 25% of the events do not express gene-of-interest mRNA and if the total cost of carrying out the ELISA is $10, the Western immunoassay is $50 and RT-PCR is $10, then:

AVI ELISA=(30)/(10)(500)=0.006

AVI Western=(30)/(50)(500)=0.0012

AVI RT-PCR=(25)/(10)(500)=0.005

It can be concluded from this analysis that the ELISA would be slightly preferable to RT-PCR, with both more efficient and/or effective than Western immunoassay. Thus, a staged experimental plan would provide for performing the ELISA analysis instead of the Western immunoassay or the RT-PCR analysis.

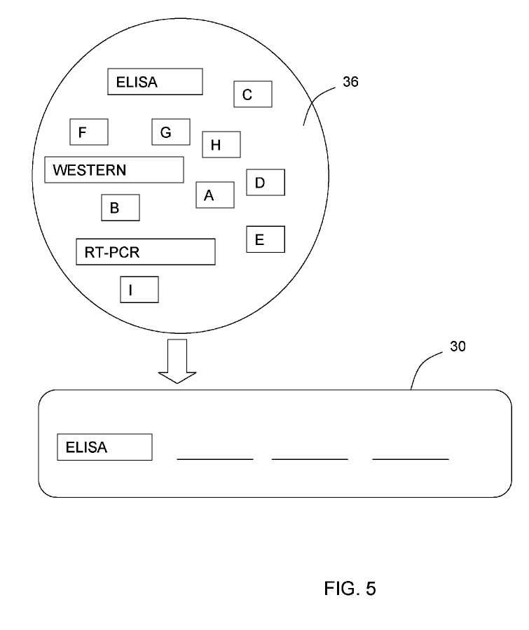

As shown in FIG. 5, suppose that within the set of possible experiments, are included ELISA, Western immunoassay, and RT-PCR. Here, based on the AVI analysis, ELISA is selected as a first experimental stage of the staged experimental plan.

### EXAMPLE 2

In this example, a more complex scenario is outlined wherein it is recognized that the timing of performing a particular experiment affects the efficiency. One wants to assess the best timing (T0 or T2 generation) for conducting Southern analysis to assess integrated transgene complexity (only simple insertions are to be commercialized), PCR-based evaluation of the presence/absence of T-DNA backbone (transformation was Agrobacterium-mediated), and understand the relative impacts of conducting qualitative transgene phenotype analyses on T0 plants vs. replicated quantitative analyses on T2 plants. Assume that the cost of Southern analysis is $100, the cost of T-DNA backbone analysis is $10, the cost of single plant ±transgene phenotype analysis is $5 and the cost of replicated quantitative T2 transgene phenotype analysis is $100. Southern analysis can be expected to reduce the event pool by 50% at the T0 generation and by 30% at the T2 generation. T-DNA backbone analysis can be expected to reduce the event pool by 30% at either generation. Qualitative T0 phenotype analysis can be expected to reduce the event pool by 20%, whereas a replicated quantitative analysis at the T2 generation can be expected to reduce the event pool by 40%. A T0 event pool of 500 events and a T2 pool of 200 events (i.e. T2 calculations assume that 60% of the T0 pool of events has been eliminated) will be used for calculations.

| | T0 | T2 |
|---|---|---|
| AVI Southern | (50)/(100) (500) = 0.001 | (30)/(100)(200) = 0.0015 |
| AVI Qual T0 +/− | (20)/(20) (500) = 0.002 | NA |
| Efficacy | | |
| AVI Quant Efficacy | NA | (40)/(100) (200) = 0.002 |
| AVI T-DNA | (30)/(10) (500) = 0.006 | (30)/(10) (200) = 0.015 |
| Backbone | | |

Several conclusions can be drawn from these calculations:
a. The AVI for T0 assays is T-DNA backbone >± efficacy> Southern analysis. Therefore, one would likely conduct the first two assessments at this stage.
b. The relatively high cost of Southern analysis and the ability to use less costly tools at the T0 generation support delaying Southern analysis to the T2 generation.
c. The lack of difference between qualitative T0 ± efficacy and quantitative T2 efficacy evaluations support conducting ± efficacy evaluation at the T0 generation.
d. Although conducting the T-DNA backbone screen at the T2 generation would appear to have a higher AVI than conducting the assessment at the T0 generation, that is only due to the assumption that 60% of the T0 event pool has been eliminated. Clearly this can only take place if some evaluations take place before the T2 generations and since, as noted in (a), the T-DNA backbone assessment is the most useful T0 screen, it should be used at the earlier generation.

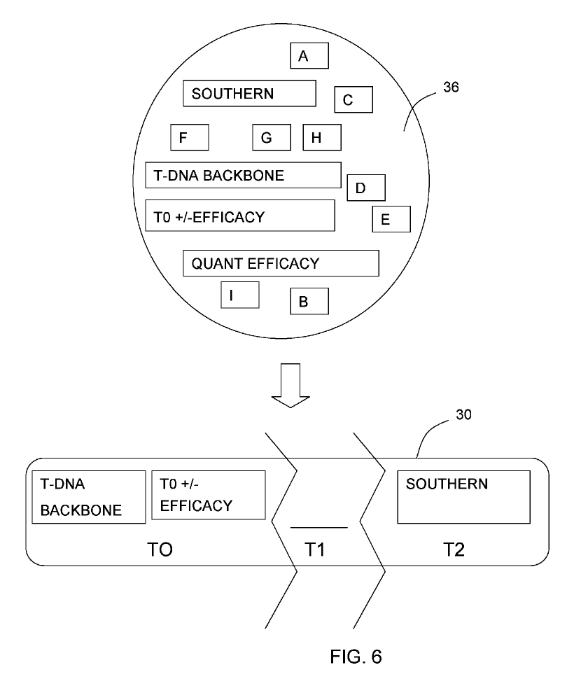

FIG. 6 illustrates one manner in which a staged experimental plan can be formed using the AVI analysis. As shown in FIG. 5, the staged experimental plan provides for conducting a T-DNA backbone screen at the T0 generation, the qualitative T0 ± at the T0 generation, and the Southern analysis at the T2 generation. Thus, the experimental stages are ordered as shown. Of course, any number of additional experimental stages may be performed at the T0, T1, or T2 generations.

Details of any plan will vary depending on the trait(s) desired in a final trait product. For example, while some phenotypic analysis of certain herbicide resistance traits can be done using T0 plants (plants directly regenerated from tissue culture), no phenotypic analysis of a grain trait such as modified protein or oil content can be done until T1 seed (seed from T0 plants that, following germination, will give rise to T1 plants) is available.

Thus, a method for identifying and selecting commercial-quality transgenic plant event from a set of transgenic plant events has been provided. Various embodiments, options, and alternatives have been discussed; however, the present invention is not to be limited to the specific examples provided herein or combinations thereof, as the present invention contemplates that any number of characteristics may be of interest for a particular type of product, any number of types of tests may be used to procure data for use in assessing characteristics, and any number of optimization methods may be used.

What is claimed is:

1. A method comprising:
transforming plant cells to produce multiple transgenic plant events;
determining a desired set of characteristics for a commercial-quality transgenic plant product;
identifying data for assessing the desired set of characteristics;
forming a staged experimental plan for generating the data;
executing the staged experimental plan to select at least one event for commercialization.

2. The method of claim 1 wherein the staged experimental plan comprises a plurality of stages for collecting and analyzing data and a plurality of gates, with each of the plurality of gates being a decision point.

3. The method of claim 2 wherein arrangement of the plurality of stages and the plurality of gates is at least partially based on at least one of availability of biological material at different stages, cost of biological testing, accuracy of biological tests at different stages and combinations thereof.

4. The method of claim 3 wherein cost effectiveness of the biological tests is at least partially determined using an economic model incorporating at least direct material and labor costs to conduct the biological tests and the predicted impact of the tests on the number of events that advance to the next stage.

5. The method of claim 4 wherein the economic model comprises assessing the cost effectiveness of biological tests by dividing the impact of the test by the total cost of the testing.

6. The method of claim 4 wherein the economic model uses a weighted index value.

7. The method of claim 4 wherein the economic model comprises an assessment value index.

8. The method of claim 1 further comprising modifying ordering of the tests such that the total cost of testing is minimized.

9. The method of claim 8 wherein the modifying the ordering of the tests occurs after one or more of the tests is performed.

10. The method of claim 1 wherein the product is a seed product.

11. The method of claim 1 wherein the desired characteristics are drought resistance, nitrogen utilization, enhanced tolerance of abiotic stresses, disease, insect, nematode or herbicide resistance, improved seed quality, yield, firmness, acidity content, sugar content, texture, modified flower senescence, improved rate of growth, traits for commercial processing, enhanced digestibility, or nutritional enhancements.

12. A method comprising:
transforming plant cells to produce multiple transgenic plant events;
determining a desired set of characteristics for a commercial-quality transgenic plant product;
identifying data for assessing the desired set of characteristics;
determining a staged experimental plan for generating the data, the staged experimental plan at least partially determined based on an economic model incorporating costs associated with biological tests and predicted impact of the biological tests on a number of the transgenic plant events that advance to a next stage;
executing the staged experimental plan to select at least one event for commercialization.
